(19) United States
(12) Patent Application Publication
(10) Pub. No.: US 2004/0024027 A1
(43) Pub. Date: Feb. 5, 2004

(54) 5(4-(2-(n-methyl-n-(2-pyridyl)amino)ethoxy)benzyl)thiazolidine-2,4-dione hydriodide as pharmaceutical

(76) Inventors: Andrew Simon Craig, Kent (GB); Tim Chien Ting Ho, Kent (GB); Michael John Millan, Kent (GB)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/297,568

(22) PCT Filed: Jun. 8, 2001
(86) PCT No.: PCT/GB01/02545

(30) Foreign Application Priority Data
Jun. 18, 2000 (GB) 0014005.3

(51) Int. Cl. C07D 417/02; A61K 31/4439

(52) U.S. Cl. 514/342; 546/269.7

(57) ABSTRACT

A novel pharmaceutical compound 5-[4-[2-(N-methyl-N-(2-pyridyl)amino)ethoxy]benzyl]thiazolidine-2,4-dione hydriodide or a solvate thereof, a process for preparing such a compound, a pharmaceutical composition comprising such a compound and the use of such a compound in medicine

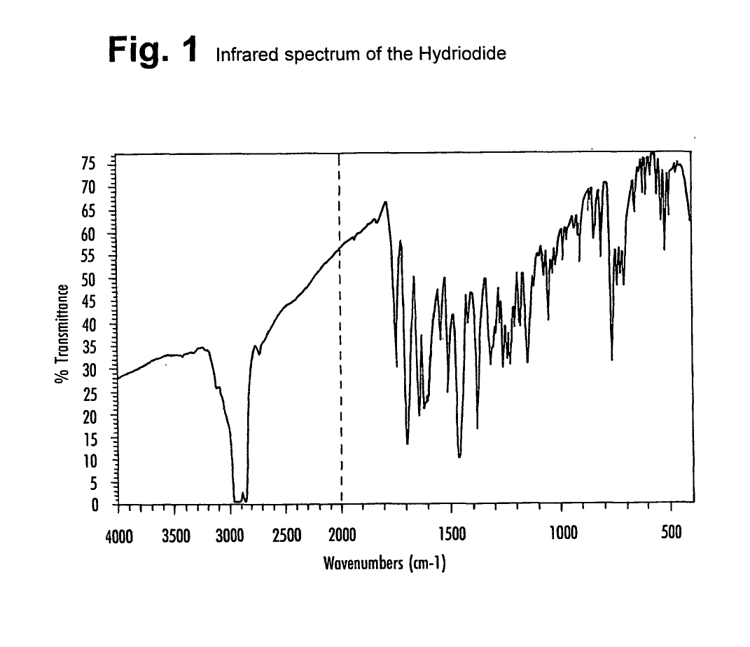

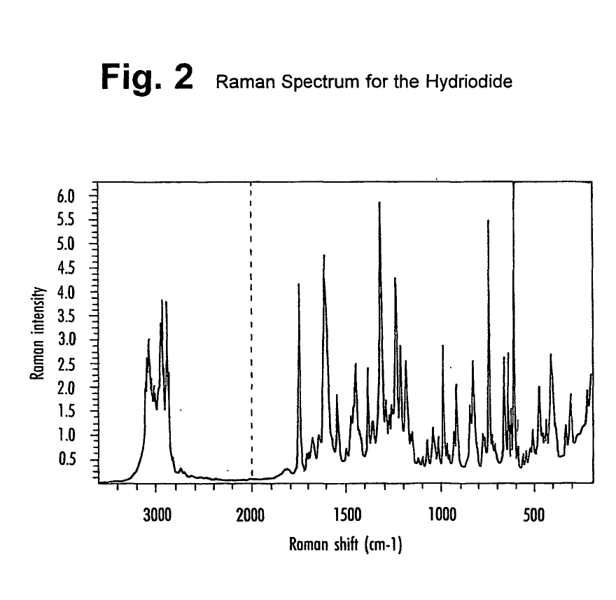

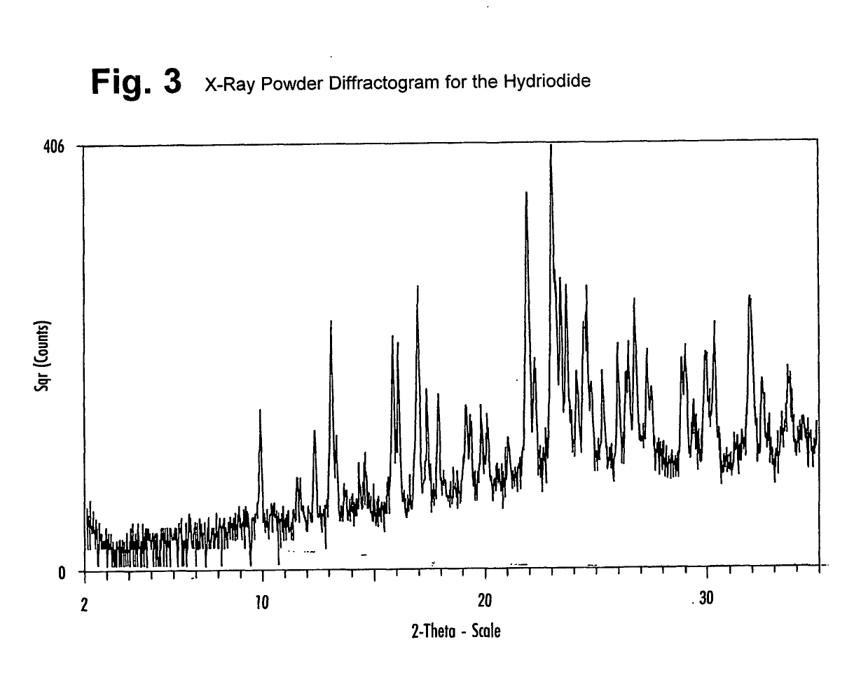

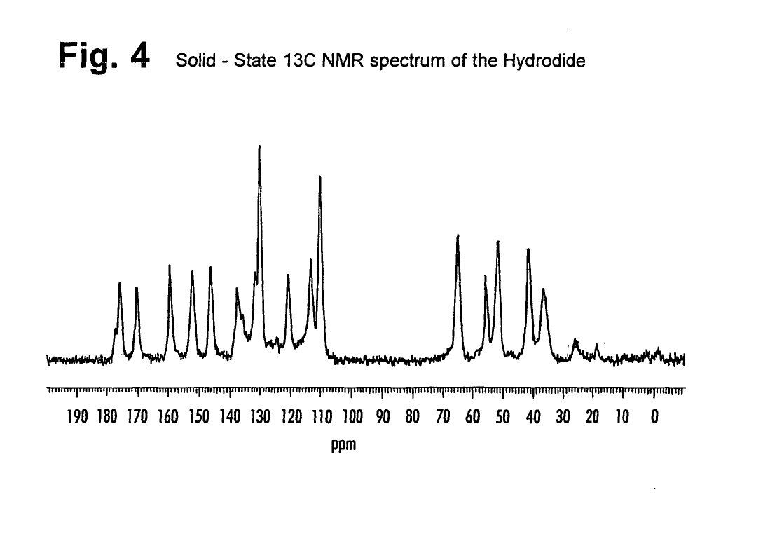

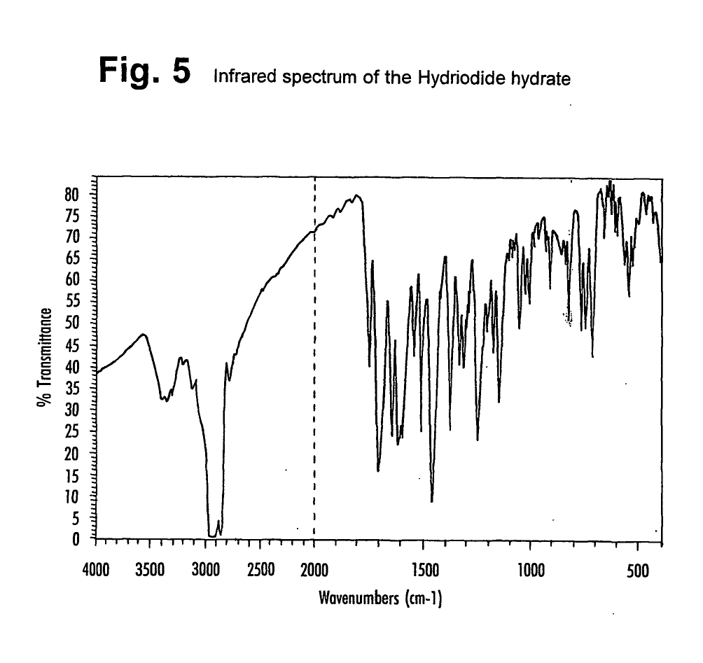

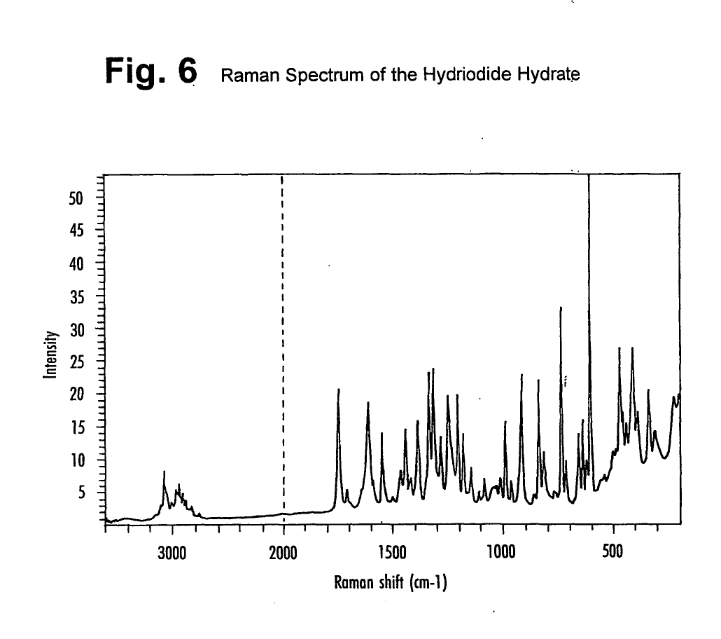

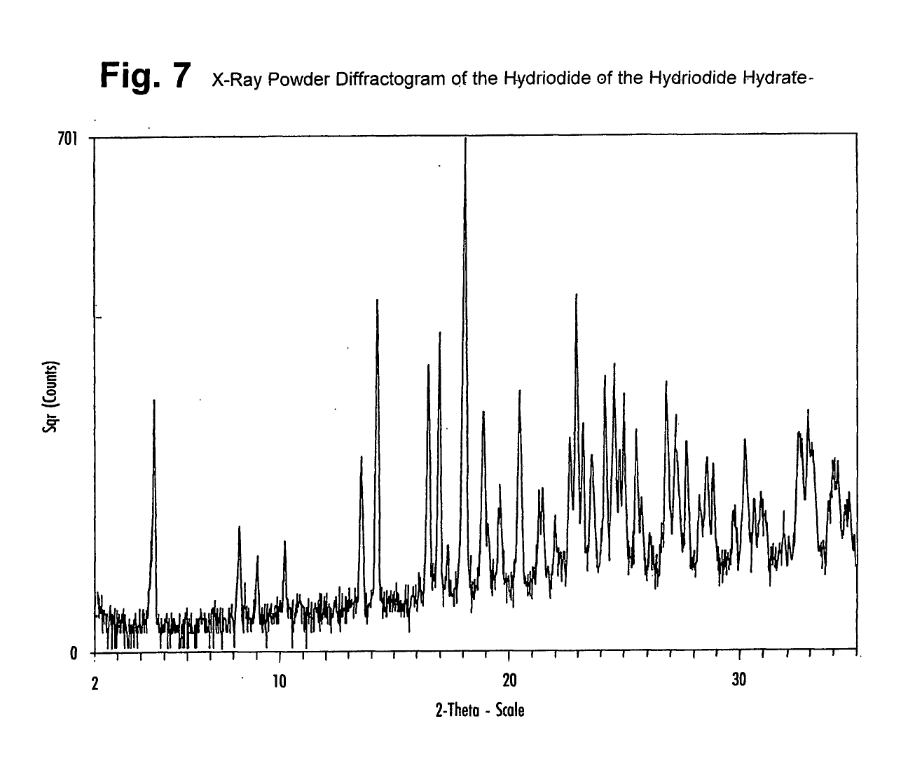

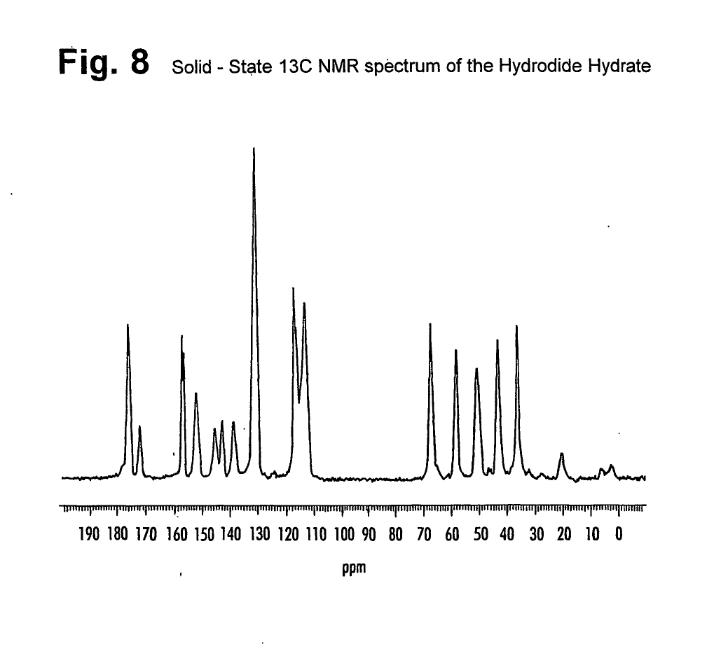

This invention relates to a novel pharmaceutical, to a process for the preparation of the pharmaceutical and to the use of the pharmaceutical in medicine.

European Patent Application, Publication Number 0,306,228 relates to certain thiazolidinedione derivatives disclosed as having hypoglycaemic and hypolipidaemic activity. The compound of example 30 of EP 0,306,228 is 5-[4-[2-(N-methyl-N-(2-pyridyl)amino)ethoxy]benzyl]thiazolidine-2,4-dione (hereinafter also referred to as “Compound (I)”).

International Patent Application, Publication Number WO94/05659 discloses certain salts of the compounds of EP 0,306,228, including salts formed from mineral acids such as hydrobromic, hydrochloric and sulphuric acids, and organic acids, such as methanesulphonic, tartaric and, in particular, maleic acid salts.

It has now been discovered that Compound (I) forms a novel hydriodide salt (hereinafter also referred to as the “Hydriodide”) that is particularly stable and hence is suitable for bulk preparation and handling. The Hydriodide also has a high melting point and possesses good bulk flow properties The Hydriodide is therefore surprisingly amenable to large scale pharmaceutical processing and especially to large scale miling.

The novel salt can be prepared by an efficient, economic and reproducible process particularly suited to large-scale preparation.

The novel Hydriodide also has useful pharmaceutical properties and in particular it is indicated to be useful for the treatment and/or prophylaxis of diabetes mellitus, conditions associated with diabetes mellitus and certain complications thereof.

Accordingly, the present invention provides 5-[4-[2-(N-methyl-N-(2-pyridyl)amino)ethoxy]benzyl]thiazolidine-2,4-dione hydriodide or solvate thereof.

Suitably, the Hydriodide is a monohydriodide.

A suitable solvate is a Hydriodide Hydrate (the “Hydriodide Hydrate”), for example a monohydrate.

In one suitable embodiment, there is provided a Hydriodide characterised by

(i) an infrared spectrum containing peaks at about 1272, 905, 810 and 803 cm⁻¹; and/or

(ii) a Raman spectrum containing peaks at about 2925, 1211, 825 and 658 cm⁻¹; and/or

(iii) a solid-state ¹³C NMR spectrum containing peaks at about 55.6, 64.8, 109.9, 120.5 and 159.3 ppm.

In one suitable embodiment, there is provided a Hydriodide Hydrate characterised by

(i) an infrared spectrum containing peaks at about 3357, 1333, 1245 and 714 cm⁻¹; and/or

(ii) a Raman spectrum containing peaks at about 1334, 1248, 1280, 1206 cm⁻¹; and/or

(iii) a solid-state ¹³C NMR spectrum containing peaks at about 43.3, 58.0, 67.5, 117.3 and 142.9 ppm.

In one favoured aspect, the Hydriodide provides an infrared spectrum substantially in accordance with FIG. I.

In one favoured aspect, the Hydriodide provides a Raman spectrum substantially in accordance with FIG. II.

In one favoured aspect, the Hydriodide provides an X-Ray powder diffraction pattern (XRPD) substantially in accordance with FIG. III.

In one favoured aspect, the Hydriodide provides a solid-state ¹³C NMR spectrum substantially in accordance with FIG. IV.

In one favoured aspect, the Hydriodide Hydrate provides an infrared spectrum substantially in accordance with FIG. V.

In one favoured aspect, the Hydriodide Hydrate provides a Raman spectrum substantially in accordance with FIG. VI.

In one favoured aspect, the Hydriodide Hydrate provides an X-Ray powder diffraction pattern (XRPD) substantially in accordance with FIG. VII.

In one favoured aspect, the Hydriodide Hydrate provides a solid-state NMR spectrum substantially in accordance with FIG. VIII.

It is particularly preferred that the Hydriodide has a melting point within the range of from 157 to 165° C., especially 160 to 167° C., for example 165° C.

Also the Hydiodide has a Tₒₙₛₑₜ within the range of from 160 to 165° C., for example 163.5° C.

Thus in a preferred aspect, the the Hydriodide is characterised in that it provides two or more of:

(i) an infrared spectrum substantially in accordance with FIG. I;

(ii) a Raman spectrum substantially in accordance with FIG. II;

(iii) an X-Ray powder diffraction pattern (XRPD) substantially in accordance with Table 1 or FIG. III;

(iv) a solid-state ¹³C NMR spectrum substantially in accordance with FIG. IV; and

(v) a melting point within the range of from 157 to 165° C., especially 160 to 165° C., for example 163° C.

Thus in a preferred aspect, the the Hydriodide Hydrate is characterised in that it provides two or more of:

(i) an infrared spectrum substantially in accordance with FIG. V;

(ii) a Raman spectrum substantially in accordance with FIG. VI;

(iii) an X-Ray powder diffraction pattern (XRPD) substantially in accordance with Table 2 or FIG. VII; and

(iv) a solid-state ¹³C NMR spectrum substantially in accordance with FIG. VIII.

The present invention encompasses the Hydriodide or solvate thereof isolated in pure form or when admixed with other materials.

Thus in one aspect there is provided the Hydriodide or solvate thereof in isolated form.

In a further aspect there is provided the Hydriodide or solvate thereof in pure form.

In yet a further aspect there is provided the Hydriodide or solvate thereof in crystalline form.

Also, the invention provides the Hydriodide or solvate thereof in a solid pharmaceutically acceptable form, such as a solid dosage form, especially when adapted for oral administration.

Moreover, the invention also provides the Hydriodide or solvate thereof in a pharmaceutically acceptable form, especially in bulk form, such form being particularly capable of being milled.

Furthermore, the invention provides the Hydriodide or solvate thereof in a pharmaceutically acceptable form, especially in bulk form, such form having good flow properties, especially good bulk flow properties.

As indicated the invention includes solvates of the Hydriodide: One such solvate is a hydrate, in particular a monohydrate.

The invention also provides a process for preparing the Hydriodide or solvate thereof, characterised in that 5-[4-[2-(N-methyl-N-(2-pyridyl)amino)ethoxy]benzyl]thiazolidine-2,4dione (Compound (I)), or a salt thereof, preferably dispersed or dissolved in a suitable solvent, is reacted with a source of hydrogen iodide and thereafter, if required, a solvate of the Hydriodide is prepared; and the Hydriodide or solvate thereof is recovered.

A suitable solvent is an alkanol for example propan-2-ol, or a hydrocarbon, such as toluene, a ketone, such as acetone, an ester, such as ethyl acetate, an ether such as tetrahydrofuran or tertiary-butyl methyl ether, a nitrile such as acetonitrile, or a halogenated hydrocarbon such as dichloromethane, or water, or mixtures thereof. Further suitable solvents include organic acids, such as acetic acid.

Conveniently, the source of hydrogen iodide is provided by an aqueous solution of hydrogen iodide, for example a 55% solution in water. Alternatively, the source of hydrogen iodide is a solution of hydrogen iodide in an appropriate solvent, suitably the reaction solvent, for example propan-2-ol.

An alternative source of hydrogen iodide is provided by a base salt of hydriodic acid for example ammonium iodide, or the hydriodic acid salt of an amine, for example ethylamine or diethylamine.

The reaction may be carried out at ambient temperature or at an elevated temperature, for example at the reflux temperature of the solvent, although any convenient temperature that provides the required product may be employed.

Solvates of the Hydriodide are prepared according to conventional procedures. For example, when the solvate is a hydrate the Hydriodide may be treated with water. Alternatively, the reaction between Compound (I) and the source of hydrogen iodide may be carried out in water or a solvent mixture comprised substantially of water.

Recovery of the required compound generally comprises crystallisation from an appropriate solvent, conveniently the reaction solvent, usually by cooling to a temperature in the range of from 0C. to 60° C., for example 21° C. For example the Hydriodide may be crystallised from an ether such as tetrahydrofuran or tert-butylmethyl ether, or a hydrocarbon such as toluene, or an organic acid such as acetic acid, or water, or a mixture thereof. Alternatively the solvent may be removed under vacuum to provide the required product.

In one preferred form the recovery comprises initial cooling to a first temperature, such as a temperature in the range of from 40-60° C., thereby allowing initiating crystallisation and thereafter cooling to a second temperature, suitably in the range of from 0 to 25° C., to complete crystallisation.

Crystallisation can also be initiated by seeding with crystals of the Hydriodide or solvate thereof but this is not essential.

Compound (I) is prepared according to known procedures, such as those disclosed in EP 0,306,228 and WO94/05659. The disclosures of EP 0,306,228 and WO94/05659 are incorporated herein by reference.

When used herein the term “Tₒₙₛₑₜ” is generally determined by Differential Scanning Calorimetry and has a meaning generally understood in the art, as for example expressed in Pharmaceutical Thermal Analysis, Techniques and Applications”, Ford and Timmins, 1989 as “The temperature corresponding to the intersection of the pre-transition baseline with the extrapolated leading edge of the transition”.

When used herein in respect of certain compounds the term “good flow properties” is suitably characterised by the said compound having a Hausner ratio of less than or equal to 1.5, especially of less than or equal to 1.25.

“Hausner ratio” is an art accepted term.

When used herein the term ‘prophylaxis of conditions associated with diabetes mellitus’ includes the treatment of conditions such as insulin resistance, impaired glucose tolerance, hyperinsulinaemia and gestational diabetes.

Diabetes mellitus preferably means Type II diabetes mellitus.

Conditions associated with diabetes include hyperglycaemia and insulin resistance and obesity. Further conditions associated with diabetes include hypertension, cardiovascular disease, especially atherosclerosis, certain eating disorders, in particular the regulation of appetite and food intake in subjects suffering from disorders associated with under-eating, such as anorexia nervosa, and disorders associated with over-eating, such as obesity and anorexia bulimia. Additional conditions associated with diabetes include polycystic ovarian syndrome and steroid induced insulin resistance.

The complications of conditions associated with diabetes mellitus encompassed herein includes renal disease, especially renal disease associated with the development of Type II diabetes including diabetic nephropathy, glomerulonephritis, glomerular sclerosis, nephrotic syndrome, hypertensive nephrosclerosis and end stage renal disease.

As mentioned above the compound of the invention has useful therapeutic properties: The present invention accordingly provides the Hydriodide or solvate thereof for use as an active therapeutic substance.

More particularly, the present invention provides the Hydriodide or solvate thereof for use in the treatment and/or prophylaxis of diabetes mellitus, conditions associated with diabetes mellitus and certain complications thereof.

The Hydriodide or solvate thereof may be administered per se or, preferably, as a pharmaceutical composition also comprising a pharmaceutically acceptable carrier. Suitable methods for formulating the Hydriodide or solvate thereof are generally those disclosed for Compound (I) in the above mentioned publications.

Accordingly, the present invention also provides a pharmaceutical composition comprising the Hydriodide or solvate thereof and a pharmaceutically acceptable carrier therefor.

The Hydriodide or solvate thereof is normally administered in unit dosage form.

The active compound may be administered by any suitable route but usually by the oral or parenteral routes. For such use, the compound will normally be employed in the form of a pharmaceutical composition in association with a pharmaceutical carrier, diluent and/or excipient, although the exact form of the composition will naturally depend on the mode of administration.

Compositions are prepared by admixture and are suitably adapted for oral, parenteral or topical administration, and as such may be in the form of tablets, capsules, oral liquid preparations, powders, granules, lozenges, pastilles, reconstitutable powders, injectable and infusable solutions or suspensions, suppositories and transdermal devices. Orally administrable compositions are preferred, in particular shaped oral compositions, since they are more convenient for general use.

Tablets and capsules for oral administration are usually presented in a unit dose, and contain conventional excipients such as binding agents, fillers, diluents, tabletting agents, lubricants, disintegrants, colourants, flavourings, and wetting agents. The tablets may be coated according to well known methods in the art.

Suitable fillers for use include cellulose, mannitol, lactose and other similar agents. Suitable disintegrants include starch, polyvinylpyrrolidone and starch derivatives such as sodium starch glycollate. Suitable lubricants include, for example, magnesium stearate. Suitable pharmaceutically acceptable wetting agents include sodium lauryl sulphate.

Solid oral compositions may be prepared by conventional methods of blending, filling, tabletting or the like. Repeated blending operations may be used to distribute the active agent throughout those compositions employing large quantities of fillers. Such operations are, of course, conventional in the art.

Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethyl cellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or colouring agents.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the active compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the active compound is suspended in the vehicle instead of being dissolved and sterilised by exposure to ethylene oxide before suspending in the sterile vehicle. Advantageously, a surfactant or wetting agent is included in the composition to facilitate uniform distribution of the active compound.

As is common practice, the compositions will usually be accompanied by written or printed directions for use in the medical treatment concerned.

As used herein the term ‘pharmaceutically acceptable’ embraces compounds, compositions and ingredients for both human and veterinary use: for example the term ‘pharmaceutically acceptable salt’ embraces a veterinarily acceptable salt.

The present invention further provides a method for the treatment and/or prophylaxis of diabetes mellitus, conditions associated with diabetes mellitus and certain complications thereof, in a human or non-human mammal which comprises administering an effective, non-toxic, amount of Hydriodide or solvate thereof to a human or non-human mammal in need thereof.

Conveniently, the active ingredient may be administered as a pharmaceutical composition hereinbefore defined, and this forms a particular aspect of the present invention.

In a further aspect the present invention provides the use of Hydriodide or solvate thereof for the manufacture of a medicament for the treatment and/or prophylaxis of diabetes mellitus, conditions associated with diabetes mellitus and certain complications thereof.

In the treatment and/or prophylaxis of diabetes mellitus, conditions associated with diabetes mellitus and certain complications thereof the Hydriodide or solvate thereof may be taken in amounts so as to provide Compound (I) in suitable doses, such as those disclosed in EP 0,306,228, WO94/05659 or WO98/55 122.

No adverse toxicological effects are indicated in the above mentioned treatments for the compounds of the invention.

The following examples illustrate the invention but do not limit it in anyway.

### Example 1

5-[4-[2-(N-Methyl-N-(2-pyridyl)amino)ethxy]benzyl]thiazolidine-2,4-dione Hydriodide

A mixture of 5-[4-[2-(N-Methyl-N-(2-pyridyl)amino)ethoxy]benzyl]thiazolidine-2,4-dione (1.0 g) and propan-2-ol (50 ml) was stirred and heated to reflux for 10 minutes at which point a clear solution was observed. Hydriodic acid (0.36 g, 55% solution in water) was added to the reaction mixture which was stirred for 5 minutes at reflux and then cooled to 21° C. The solvent was removed under reduced pressure (25° C.) to give 5-[4-[2(N-Methyl-N-(2-pyridyl)amino)ethoxy]benzyl]thiazolidine-2,4dione hydriodide as a crystalline solid.

1H-NMR (d6-DMSO): consistent with the Hydriodide

### Example 2

5-[4-[2-(N-Methyl-N-(2-pyridyl)amino)ethoxy]benzyl]thiazolidine-2,4-dione Hydriodide

Hydriodic acid (1.79 g, 55% solution in water) was added to a stirred solution of 5-[4-[2-(N-Methyl-N-(2-pyridyl)amino)ethoxy]benzyl]thiazolidine-2,4dione (5.0 g) in THF (50 ml) at 21° C., and the reaction mixture was stirred for 30 minutes at 21° C. The reaction mixture was heated to 60° C. for 1 hour, cooled to 21° C. and the solvent removed under reduced pressure. Toluene (50 ml) was added to the residue and the mixture stirred and then solvent was removed under reduced pressure to give 5-[4-[2-(N-Methyl-N-(2-pyridyl)amino)ethoxy]benzyl]thiazolidine-2,4-dione hydriodide (6.7 g) as a crystalline solid.

### Example 3

5-[4-[2-(N-Methyl-N-(2-pyridyl)amino)ethoxy]benzyl]thiazolidine-2,4-dione Hydriodide

A suspension of 5-[4-[2-(N-Methyl-N-(2-pyridyl)amino)ethoxy]benzyl]thiazolidine-2,4-dione (5.0 g) in toluene (50 mL) was heated to 50° C. before hydriodic acid (1.91 mL, 55% solution in water) was added. The reaction temperature was raised to 110° C. and the mixture stirred for 15 minutes. The mixture was cooled to 21° C. and the solid collected by filtration and dried under vacuum over phosphorus pentoxide for 16 hours to give 5-[4-[2N-Methyl-N-(2-pyridyl)amino)ethoxy]benzyl]thiazolidine-2,4-dione hydriodide (6.8 g) as a crystalline solid.

### Example 4

5-[4-[2-(N-Methyl-N-(2pyridyl)amino)ethoxy]benzyl]thiazolidine-2,4-dione Hydriodide

A mixture of 5-[4-[2-(N-Methyl-N-(2-pyridyl)amino)ethoxy]benzyl]thiazolidine-2,4-dione (5.0 g) and t-butyl methyl ether (50 mL) was stirred and heated to reflux. Hydriodic acid (1.91 mL, 55% in water) was added and the reaction mixture was heated at reflux for 1 hour. The mixture was cooled to 21° C. and the solid was recovered by filtration and washed with t-butyl methyl ether. The product was dried under vacuum at 21° C. for 16 hours to give 5-[4-[2-(N-Methyl-N-(2-pyridyl)amino)ethoxy]benzyl]thiazolidine-2,4-dione hydriodide (6.6 g) as a crystalline solid.

### Example 5

5-[4-[2-(N-Methyl-N-(2-pyridyl)amino)ethoxyl]benzyl]thiazolidine-2,4-dione Hydriodide

A mixture of 5-[4-[2-(N-Methyl-N-(2-pyridyl)amino)ethoxy]benzyl]thiazolidine-2,4-dione (20.0 g) and acetic acid (200 mL) was stirred and heated to 100° C. at which point hydriodic acid (7.67 mL, 55% solution in water) was added. The clear solution was cooled to 50° C. over 30 minutes and was then seeded with the product of Example 4. After stirring for 15 minutes at 50° C. the mixture was cooled to 21° C. over a period of 30 minutes and stirred at 21° C. for a further 30 minutes. The solid was collected by filtration, washed with water (2×40 mL) and dried under vacuum at 50° C. to give 5-[4-[2-(N-Methyl-N-(2-pyridyl)amino)ethoxy]benzyl]thiazolidine-2,4-dione hydriodide (21.6 g) as a crystalline solid.

### CHARACTERISING DATA FOR THE HYDRIODIDE RECORDED FOR THE PRODUCT OF EXAMPLE 1

The infrared absorption spectrum of a mineral oil dispersion of the product was obtained using a Nicolet 710 FT-IR spectrometer at 2 cm⁻¹ resolution (FIG. I). Data were digitised at 1 cm-1 intervals. Bands were observed at: 1743, 1696, 1643, 1616, 1543, 1512, 1462, 1418, 1378, 1313, 1272, 1259, 1237, 1225, 1205, 1183, 1177, 1145, 1069, 1050, 1031, 1016, 986, 968, 905, 842, 810, 803, 763, 737, 722, 708, 656, 619, 603, 584, 557, 537, 520, 502 cm⁻¹.

The infrared spectrum of the solid product was recorded using Perkin-Elmer Spectrum One FT-IR spectrometer fitted with a universal ATR accessory. Bands were observed at: 3027, 2970, 2875, 1743, 1695, 1642, 1615, 1601, 1544, 1512, 1443, 1419, 1380, 1361, 1314, 1289, 1272, 1258, 1237, 1224, 1204, 1184, 1177, 1144, 1114, 1069, 1050, 1031, 1016, 986, 968, 951, 933, 915, 905, 859, 841, 810, 803, 761, 737, 722, 706, 656 cm-1.

The Raman spectrum of the product (FIG. II) was recorded with the sample in an NMR tube using a Nicolet 960 E.S.P. FT-Raman spectrometer, at 4 cm⁻¹ resolution with excitation from a Nd:V04 laser (1064 nm) with a power output of 400 mW. Bands were observed at: 3085, 3063, 2947, 2925, 2879, 2858, 1746, 1670, 1609, 1545, 1443, 1382, 1358, 1316, 1290, 1236, 1211, 1182, 1070, 1041, 1015, 986, 968, 929, 915, 843, 825, 739, 658, 636, 621, 604, 503, 470, 431, 405, 331, 303, 219, 112 cm⁻¹.

The X-Ray Powder Diffractogram pattern of the product (FIG. III) was recorded using the following acquisition conditions: Tube anode: Cu, Generator tension: 40 kV,

Generator current: 40 mA, Start angle: 2.0° 2θ, End angle: 35.0° 2θ, Step size: 0.02° 2θ, Time per step: 2.5 seconds. Characterisaic XRPD angles and relative intensities are recorded in Table 1.

TABLE 1

| | |
|---|---|
| Angle | Rel. Intensity |
| 2-Theta° | % |

| | |
|---|---|
| 9.9 | 14.3 |
| 11.6 | 4.7 |
| 12.4 | 10.8 |
| 13.1 | 35 |
| 14.6 | 6.7 |
| 15.9 | 30.5 |
| 16.1 | 28.8 |
| 17.0 | 44.3 |
| 17.4 | 18 |
| 17.9 | 17 |
| 19.2 | 15 |
| 19.9 | 14.8 |
| 20.1 | 13.1 |
| 21.1 | 9.6 |
| 22.0 | 79.1 |
| 22.3 | 24.6 |
| 23.1 | 100 |
| 23.5 | 46.8 |
| 23.7 | 44.6 |
| 24.2 | 21.4 |
| 24.6 | 36.5 |
| 25.4 | 21.7 |
| 26.1 | 28.3 |
| 26.5 | 28.8 |
| 26.8 | 40.4 |
| 27.4 | 26.6 |
| 27.5 | 18 |
| 29.0 | 18.7 |
| 29.5 | 15.5 |
| 30.0 | 26.1 |
| 30.4 | 33.7 |
| 32.0 | 40.4 |
| 32.6 | 19.5 |
| 33.8 | 20.9 |

The solid-state NMR spectrum of the product (FIG. IV) was recorded on a Bruker AMX360 instrument operating at 90.55 MHz: The solid was packed into a 4 mm zirconia MAS rotor fitted with a Kel-F cap and the rotor spun at ca. 10 kHz. The ¹³C MAS spectrum was acquired by cross-polarisation from Hartmann-Hahn matched protons (CP contact time 3ms, repetition time 15 s) and protons were decoupled during acquisition using a two-pulse phase modulated (TPPM) composite sequence. Chemical shifts were externally referenced to the carboxylate signal of glycine at 176.4 ppm relative to TMS and were observed at: 36.5, 41.3, 51.6, 55.6, 64.8, 109.9, 113.3, 120.5, 129.9, 131.5, 137.2, 146.1, 152.1, 159.3, 170.4, 175.5 ppm.

### PROPERTIES OF THE HYDRIODIDE RECORDED FOR THE PRODUCT OF EXAMPLE 5

Solid State Stability of the Hydriodide

1) The solid state stability of the drug substance was determined by storing approximately 1.0 g of the material in a glass bottle at a) 40° C./75% Relative Humidity (RH), open exposure, for 1 month and b) at 50° C., closed, for 1 month. The material was assayed by HPLC for final content and degradation products in both cases.

a) 40° C./75% RH: No significant degradation observed (HPLC assay 97% initial).

b) 50° C.: No significant degradation observed (HPLC assay 97% initial).

2) A weighed sample of the Hydriodide (0.105 g) was placed in a sealed container under a 75% Relative Humidity atmosphere (saturated sodium chloride solution) at 21° C. for 96 hours. The sample was re-weighed and the infrared spectrum of the product recorded.

Sample weight: no weight gain

Infrared spectrum: no change

Flow Properties of the Hydriodide:

The ratio between the bulk density and the tapped bulk density (Hausner Ratio) of the Hydrobromide was determined using standard methods (“Pharmaceutics—The Science of Dosage Form Design”, editor M. Aulton, 1988, published by: Churchill Livingstone). Hausner Ratio: 1.1

Tₒₙₛₑₜ of the Hydriodide

The Tₒₙₛₑₜ of the drug substance was determined by Differential Scanning Calorimetry using a Perkin-Elmer DSC7 apparatus. Tₒₙₛₑₜ: 163.30° C.

Melting Point of the Hydriodide

The melting point of the drug substance was determined visually by hot stage microscopy. Mpt: 165 ° C.

### Example 6

5-[4-[2-(N-Methyl-N-(2-pyridyl)amino)ethoxy]benzyl]thiazolidine-2,4-dione hydriodide hydrate

A suspension of 5-[4-[2-(N-Methyl-N-(2-pyridyl)amino)ethoxy]benzyl]thiazolidine-2,4-dione (3.0 g) in water (90 mL) was stirred and heated to reflux and hydriodic acid (1.15 mL, 55% in water) was added, resulting in a clear solution after 5 minutes. The mixture was cooled to 55° C. and seeded with the product of Example 3 and then cooled to 21° C. over a period of approximately 1 hour. The product was collected by filtration and dried under vacuum over phosphorus pentoxide for 16 hours to give 5-[4-[2-(N-Methyl-N-(2-pyridyl)amino)ethoxy]benzyl]thiazolidine-2,4-dione hydriodide hydrate (3.86 g)

### Example 7

5-[4-[2-(N-Methyl-N-(2-pyridyl)amino)ethoxy]benzyl]thiazolidine-2,4 -dione Hydriodide Hydrate

A suspension of 5-[4-[2-(N-Methyl-N-(2-pyridyl)amino)ethoxy]benzyl]thiazolidine-2,4-dione (3.0 g, 8.39 mmol) in water (30 mL) was stirred and heated to reflux. Hydriodic acid (1.15 mL, 8.39 mmol, 55% in water) was added and the mixture stirred at reflux for 15 minutes The solution was cooled to 70° C., at which point a turbidity was observed, and the mixture warmed to 80° C., seeded with the product of Example 6 and then cooled to 21° C. The product was collected by filtration, washed with water (10 ml) and dried for 16 hours under vacuum over phosphorus pentoxide to give 5-[4-[2-(N-Methyl-N-(2-pyridyl)amino)ethoxy]benzyl]thiazolidine-2,4-dione hydriodide hydrate (3.75 g).

K-F(water): determined as 3.4 % by wt.

1H-NMR (d6-DMSO): consistent with 5-[4-[2-(N-Methyl-N-(2-pyridyl)amino)ethoxy]benzyl]thiazolidine-2,4-dione hydriodide

### Example 8

5-[4-[2-(N-Methyl-N-(2-pyridyl)amino)ethoxyl]benzyl]thiazolidine-2,4-dione hydriodide hydrate

Hydriodic acid (11.5 ml) was added to a stirred suspension of 5-[4-[2-(N-methyl-N-(2-pyridyl)amino)ethoxy]benzyl]thiazolidine-2,4-dione (30.0 g) in water (300 ml) at 80° C. The temperature was raised to reflux for 15 minutes, then cooled to 80° C. where the clear solution was seeded with the product of example 6. The stirred mixture was further cooled to 21° C. where the solid was collected by filtration, washed with water (100 ml) and dried for 20 hours under vacuum to afford 5-[4-[2-(N-methyl-N-(2-pyridyl)amino)ethoxy]benzyl]thiazolidine-2,4-dione hydriodide hydrate (40.8 g) as a pale yellow solid.

### Example 9

5-[4-[2-(N-Methyl-N-(2-pyridyl)amin)ethoxy]benzyl]thiazolidine-2,4-dione hydriodide hydrate

Hydriodic acid (1.15 ml) was added to a stirred suspension of 5-[4-[2-(N-Methyl-N-2-pyridyl)amino)ethoxy]benzyl]thiazolidine-2,4-dione (3.0 g) in water (30 ml) at reflux. The solution was held at reflux for 5 minutes and then cooled to 21° C. over approximately 90 minutes with stirring. The mixture was heated to approximately 55° C., at which point crystallisation was observed, and the stirred mixture cooled to 21° C. The solid was collected by filtration, washed with water (10 ml), and dried for 24 hours under vacuum to yield 5-[4-[2-(N-methyl-N-(2-pyridyl)amino)ethoxy]benzyl]thiazolidine-2,4-dione hydriodide hydrate (4.1 g) as a crystalline solid.

### CHARACTERISING DATA FOR THE HYDRIODIDE HYDRATE RECORDED FOR THE PRODUCT OF EXAMPLE 7

The infrared absorption spectrum of a mineral oil dispersion of the product was obtained using a Nicolet 710 FT-IR spectrometer at 2 cm⁻¹resolution (FIG. V). Data were digitised at 1 cm⁻¹ intervals. Bands were observed at: 3357, 2919, 2853, 2784, 1746, 1703, 1641, 1615, 1545, 1512, 1461, 1378, 1333, 1312, 1287, 1245, 1206, 1177, 1151, 1053, 1025, 1006, 913, 825, 766, 746, 714, 652, 559, 541, 525, 468 cm-1.

The infrared spectrum of the solid product was recorded using Perkin-Elmer Spectrum One FT-IR spectrometer fitted with a universal AIR accessory. Bands were observed at: 3400, 3361, 3312, 2780, 1746, 1700, 1641, 1608, 1596, 1545, 1512, 1461, 1442, 1421, 1379, 1332, 1312, 1287, 1243, 1206, 1177, 1151, 1052, 1025, 1006, 985, 963, 932, 913, 859, 839, 824, 765, 745, 711 cm⁻¹.

The Raman spectrum of the product (FIG. VI) was recorded with the sample in an NMR tube using a Nicolet 960 E.S.P. FT-Raman spectrometer, at 4 cm⁻¹ resolution with excitation from a Nd:V04 laser (1064 nm) with a power output of 400 mW. Bands were observed at: 3071, 2933, 2902, 1746, 1709, 1607, 1546, 1462, 1439, 1415, 1381, 1334, 1313, 1280, 1248, 1206, 1180, 1143, 1108, 1080, 1028, 1009, 988, 962, 914, 840, 819, 775, 739, 717, 654, 637, 622, 606, 468, 452, 434, 408, 386, 334, 308, 224 cm⁻¹.

The X-Ray Powder Diffractogram pattern of the product (FIG. VII) was recorded using the following acquisition conditions: Tube anode: Cu, Generator tension: 40 kV, Generator current: 40 mA, Start angle: 2.0° 2θ, End angle: 35.0° 2θ, Step size: 0.02° 2θ, Time per step: 2.5 seconds.Characteristic XRPD angles and relative intensities are recorded in Table 2.

TABLE 2

| Angl | Rel. Intensity |
|---|---|
| 2-Theta° | % |
| 4.5 | 19.3 |
| 8.2 | 6 |
| 9.0 | 2.6 |
| 10.2 | 4.6 |
| 13.5 | 14.4 |
| 14.2 | 47.4 |
| 16.5 | 31.4 |
| 17.0 | 38.8 |
| 17.3 | 3.9 |
| 18.1 | 100 |
| 18.9 | 22 |
| 19.6 | 10.7 |
| 20.5 | 26 |
| 21.4 | 9.8 |
| 22.0 | 7 |
| 22.7 | 17.3 |
| 22.9 | 48.6 |
| 23.2 | 19.7 |
| 23.6 | 14.1 |
| 24.2 | 28.7 |
| 24.6 | 31.4 |
| 25.0 | 25.1 |
| 25.6 | 18.5 |
| 25.8 | 9 |
| 26.2 | 5 |
| 26.9 | 27.5 |
| 27.3 | 21 |
| 27.8 | 16.7 |
| 28.3 | 9.1 |
| 28.6 | 14 |
| 28.9 | 13.3 |
| 29.8 | 7.4 |
| 30.3 | 16.4 |
| 30.7 | 8.7 |
| 30.9 | 9.6 |
| 31.1 | 7.4 |
| 31.9 | 7.3 |
| 32.6 | 17.5 |
| 33.0 | 21.8 |
| 33.2 | 15 |
| 34.1 | 14 |
| 34.3 | 13.4 |
| 34.7 | 9.1 |

The solid-state NMR spectrum of the product (FIG. III) was recorded on a Bruker AMX360 instrument operating at 90.55 MHz: The solid was packed into a 4 mm zirconia MAS rotor fitted with a Kel-F cap and rotor spun at ca. 10 kHz. The ¹³C MAS spectrum was acquired by cross-polarisation from Hartmann-Hahn matched protons (CP contact time 3 ms, repetition time 15 s) and protons were decoupled during acquisition using a two-pulse phase modulated (TPPM) composite sequence. Chemical shifts were externally referenced to the carboxylate signal of glycine at 176.4 ppm relative to TMS and were observed at: 36.4, 43.3, 51.0, 58.1, 67.5, 113.3, 116.5, 117.3, 131.1, 138.7, 142.9, 145.3, 152.3, 156.7, 157.4, 172.0, 175.9 ppm.

### PROPERTIES OF THE HYDRIODIDE HYDRATE RECORDED FOR THE PRODUCT OF EXAMPLE 8

Solid State Stability of the Hydriodide Hydrate

The solid state stability of the drug substance was determined by storing approximately 1.0 g of the material in a glass bottle at a) 40° C./75% Relative Humidity (RH), open exposure, for 1 month and b) at 50° C., closed, for 1 month The material was assayed by HPLC for final content and degradation products in both cases.

a) 40° C./75% RH: No significant degradation observed (HPLC assay 97% initial).

b) 50° C.: No significant degradation observed (HPLC assay 98% initial).

Tₒₙₛₑₜ of the Hydriodide Hydrate

The Tₒₙₛₑₜ was determined by Differential Scanning Calorimetry using a Perkin-Elmer DSC7 apparatus. Tₒₙₛₑₜ: 110 ° C.

Melting Point of the Hydriodide Hydrate

The melting point was detemined visually via hot stage microscopy. Mpt: 116-118° C.

1. A compound 5-[4-[2-(N-methyl-N-(2-pyridyl)amino)ethoxy]benzyl]thiazolidine-2,4dione hydriodide or a solvate thereof.

2. A compound according to claim 1, characterised in that it provides:
(i) an infrared spectrum containing peaks at about 1272, 905, 810 and 803cm⁻¹; and/or
(ii) a Raman spectrum containing peaks at about 2925, 1211, 825 and 658 cm⁻¹; and/or
(iii) a solid-state ¹³C NMR spectrum containing peaks at about 55.6, 64.8, 109.9, 120.5 and 159.3 ppm.

3. A compound according to claim 1, characterised in that it provides two or more of:
(i) an infrared spectrum substantially in accordance with FIG. I;
(ii) a Raman spectrum substantially in accordance with FIG. II;
(iii) an X-Ray powder diffraction pattern (XRPD) substantially in accordance with Table 1 or FIG. III;
(iv) a solid-state ¹³C NMR spectrum substantially in accordance with FIG. IV; and
(v) a melting point within the range of from 157 to 165° C.

4. A compound according to any one of claims 1 to 3, in purified form.

5. A compound according to any one of claims 1 to 3, in a solid dosage form.

6. A compound according to any one of claims 1 to 3, in a pharmaceutically acceptable form capable of being milled.

7. A compound according to any one of claims 1 to 3, in a pharmaceutically acceptable form having good flow properties.

8. A process for preparing 5-[4-[2-(N-methyl-N-(2-pyridyl)amino)ethoxy]benzyl]thiazolidine-2,4-dione hydriodide or a solvate thereof., characterised in that 5-[4-[2-(N-methyl-N-(2-pyridyl)amino)ethoxy]benzyl]thiazolidine-2,4-dione or a salt thereof is reacted with a source of hydrogen iodide and thereafter, if required, a solvate of the Hydriodide is prepared; and the Hydriodide or solvate thereof is recovered.

9. A pharmaceutical composition comprising 5-[4-[2-(N-methyl-N-(2-pyridyl)amino)ethoxy]benzyl]thiazolidine-2,4dione hydriodide or a solvate thereof. and a pharmaceutically acceptable carrier therefor.

10. A compound 5-[4-[2-(N-methyl-N-(2-pyridyl)amino)ethoxy]benzyl]thiazolidine-2,4dione hydriodide or a solvate thereof for use as an active therapeutic substance.

11. A use of 5-[4-[2-(N-methyl-N-(2-pyridyl)amino)ethoxy]benzyl]thiazolidine-2,4 -dione hydriodide or a solvate thereof, for the manufacture of a medicament for the treatment and/or prophylaxis of diabetes mellitus, conditions associated with diabetes mellitus and certain complications thereof.
